(19) United States
(12) Patent Application Publication
(10) Pub. No.: US 2012/0156767 A1
(43) Pub. Date: Jun. 21, 2012

(54) PCR DEVICE

(75) Inventors: Lucio RENNA, Catania (IT); Clelia GALATI, San Gragorio Di Catania (IT); Natalia Maria Rita SPINELLA, Paterno' (IT)

(73) Assignee: STMicroelectronics S.r.l., Agrate Brianza (IT)

(21) Appl. No.: 13/316,751

(22) Filed: Dec. 12, 2011

(30) Foreign Application Priority Data
Dec. 17, 2010 (IT) TO2010A001013

(51) Int. Cl. C12M 1/40; B32B 9/04

(52) U.S. Cl. 435/289.1; 428/704

(57) ABSTRACT

PCR device (1) comprising at least a chamber (2), the chamber (2) being delimited on one side by a PCR substrate (3) at least partially covered by a polyelectrolyte coating (4) forming hydrophilic regions. The polyelectrolyte coating comprises at least one layer facing the chamber (2), the layer being a negatively charged polyelectrolyte layer. Further a hydrophobic polymer layer may be applied onto the PCR substrate facing the chamber forming hydrophobic regions delimiting the hydrophilic regions. Further a method for passivating a PCR substrate comprising the step of applying a polyelectrolyte is provided for.

### PRIOR RELATED APPLICATIONS

This application claims priority to Italian Patent Application No. TO2010A 001013, filed on Dec. 17, 2010 in the name of STMicroelectronics S.r.l. and incorporated by reference in its entirety herein.

### FEDERALLY SPONSORED RESEARCH STATEMENT

Not applicable.

### REFERENCE TO MICROFICHE APPENDIX

Not applicable.

### FIELD OF THE INVENTION

The present invention relates to PCR devices for biological analysis.

### BACKGROUND OF THE INVENTION

Typical procedures for analyzing biological materials, such as nucleic acid, involve a variety of operations starting from raw material. These operations may include various degrees of cell purification, lysis, amplification or purification, and analysis of the resulting amplified or purified product.

As an example, in DNA-based blood tests the samples are often purified by filtration, centrifugation or by electrophoresis so as to eliminate all the non-nucleated cells. Then, the remaining white blood cells are lysed using chemical, thermal or biochemical means in order to liberate the DNA to be analyzed.

Next, the DNA is denatured by thermal, biochemical or chemical processes and amplified by an amplification reaction, such as PCR (polymerase chain reaction), LCR (ligase chain reaction), SDA (strand displacement amplification), TMA (transcription-mediated amplification), RCA (rolling circle amplification), and the like. The amplification step allows the operator to avoid purification of the DNA being studied because the amplified product greatly exceeds the starting DNA in the sample.

The procedures are similar if RNA is to be analyzed, but more emphasis is placed on purification or other means to protect the labile RNA molecule. RNA is usually copied into DNA (cDNA) and then the analysis proceeds as described for DNA.

Finally, the amplification product undergoes some type of analysis, usually based on sequence or size or some combination thereof. In an analysis by hybridization, for example, the amplified DNA is passed over a plurality of detectors made up of individual oligonucleotides, hereinafter called probes, that are anchored, for example, on electrodes. If the amplified DNAs are complementary to the probes, stable bonds will be formed between them and the hybridized probes can be read using a wide variety of means, including optical, electrical, mechanical, magnetic or thermal means.

Other biological molecules are analyzed in a similar way, but typically molecule purification is substituted for amplification and detection methods vary according to the molecule being detected. For example, a common diagnostic involves the detection of a specific protein by binding to its antibody or by a specific enzymatic reaction. Lipids, carbohydrates, drugs and small molecules from biological fluids are processed in similar ways.

It is known to perform both amplification and detection in integrated devices comprising a semiconductor support, for example a silicon substrate, generally including an amplification zone and a detection zone.

Due to their surface charge, silicon substrates can interact with the ingredients from biological assays, for example with PCR-mix components. This interaction can cause changes in the amplification process, and thus decrease the performance of subsequent analyses.

Two main mechanisms have been proposed to account for PCR inhibition in silicon-based PCR chip: (i) straight chemical action of the surface materials on the PCR solution and (ii) surface adsorption of one or more components of the PCR solution onto the PCR reaction walls.

The latter is particularly important in miniaturized PCR-chips due to the significant increase of the “Surface-to-Volume” Ratio (SVR) as compared to the conventional macro instrument. In fact, with the increase of SVR, the significance of surface chemistry in the PCR amplification increases and the deleterious surface interactions, e.g. surface adsorption, are even more likely to occur.

Erill et al. (I. Erill, S. Campoy, N. Erill, J. Barbé, J. Aguiló “Biochemical analysis and optimization of inhibition and adsorption phenomena in glass-silicon PCR-chips”, Sensors and Actuators B 96 (2003) 685-692) found that silicon-related materials do not seem to be potent straight chemical inhibitors of PCR, but that the PCR inhibition was mainly caused by a preferential adsorption of the DNA-polymerase onto the PCR reaction chambers, as compared with other PCR components (DNA templates, ions metal, primers, etc.). The DNA-polymerase adsorption on silicon-based PCR-chips was recently confirmed in real-time PCR studies.

Although the mechanisms that govern the inhibition of PCR by silicon-related materials are still not clearly understood, the above mentioned findings improved the understanding of the inhibitory effects displayed in silicon-based PCR-chips.

It is therefore commonly known to coat silicon substrates with a passivation layer for providing insulation and protection against undesired chemical interactions between silicon reactive groups and the biological sample. In fact, passivated substrates show reduced interaction with PCR-mix components, thus improving analysis yields.

The passivation is conducted with different techniques. In particular, the passivation method that gives the best results is the formation of a silicon dioxide layer.

Nevertheless, although the reactivity of passivated silicon substrates towards biological molecules is lower than the silicon as such, experimental tests proved that passivated silicon substrates still show a residual reactivity, which can alter the results of subsequent analyses.

Therefore, it is well known to those skilled in the art to treat passivated silicon substrates in the amplification zone with further treatments, such as deposition of silanes, in order to further reduce the reactivity of the silicon substrate. Although these treatments show good results, none of them provides a significant increase in yield as compared to the silicon dioxide alone.

Alternatively, it is known to passivate silicon substrate with Bovine Serum Albumin (BSA). However, several disadvantages have to be considered in the use of such material, such as high costs, temperature instability and storage requirements.

The same reduction in PCR analysis yields, noted for silicon-related substrates, has been also observed when the PCR is carried out on different substrates such as glass or plastics.

Thus, what is needed in the art are better methods of performing analyses such as PCR on microdevices, wherein surface chemistry of the device does not negatively impact the analysis.

### SUMMARY OF THE INVENTION

The aim of the present invention is to provide a novel PCR device that is free from the above described drawbacks.

In particular, it would be desirable to find a device comprising a PCR substrate that is stable to PCR denaturation temperatures and pHs and is able to reduce interactions between PCR-mix components and the substrate, thus improving analytical results and allowing repeated biological analyses on the same substrate.

According to the present invention this aim is obtained by a PCR device according to claim 1 and by a method of passivating PCR substrates according to claim 10.

The PCR device according to the invention comprises at least one chamber. The chamber is delimited on one side by a substrate, that may be selected from the group consisting of a semiconductor substrate, a metal substrate, a glass substrate and a polymeric substrate.

In particular, a semiconductor substrate selected from the group consisting of a silicon substrate, a silicon dioxide-passivated substrate, silicon nitride-passivated substrate or a silanized substrate may be used.

The PCR substrate is at least partially covered by a polyelectrolyte coating that faces the chamber (i.e., on its outer surface), forming hydrophilic regions.

Polyelectrolytes are polymers whose monomer units have a strong or weak, acid or basic electrolyte group. These groups dissociate in aqueous solutions, making the polymers charged.

The polyelectrolyte coating may be applied for example by immersion of the PCR substrate into a polyelectrolyte solution or by piezo-spotting techniques so as to form hydrophilic regions onto the PCR substrate.

The polyelectrolyte coating passivates the PCR substrate, demonstrating very low interaction with the PCR reactants and giving the device higher stability over PCR conditions, namely temperature and pH, when compared to Bovine Serum Albumin. Moreover, polyelectrolytes are low cost materials with tunable properties that can be controlled by balancing the positive and negative charges.

The polyelectrolytes may be applied by simple water-based processes and allow simple customization, easy storage and dispensing. Further, these materials can be easily patterned on PCR substrates.

Moreover polyelectrolytes can be synthesized to carry the desired number of charges optimizing the interaction with a specific surface.

The polyelectrolyte coating comprises at least one layer, facing the chamber, i.e. it may be a single-layer coating or a multi-layer coating. The layer facing the chamber must be a negatively charged polyelectrolyte layer, in particular selected from the group consisting of polyacrylic acid, polymethacrylic acid, poly(glutamic acid), polystyrene sulfonate, derivatives, mixtures and copolymers thereof, more in particular polyacrylic acid.

It has been shown that the presence of a negatively charged polyelectrolyte layer directly facing the chamber wherein the PCR takes place reduces interactions of the PCR substrate and PCR mix elements with the chamber surfaces, as compared with BSA.

The polyelectrolyte coating may further comprise further layers between the PCR substrate and the negatively charged polyelectrolyte layer, thus being a multi-layer coating.

The further layer may be selected from the group consisting of polyacrylic acid, polyallylamine hydrochloride, polymethacrylic acid, polylysine, poly(glutamic acid), polystyrene sulfonate, polydiallyldimethyl-ammonium chloride, derivatives, mixtures and co-polymers thereof.

The multi-layer coating may be obtained by subsequent deposition of multiple polyelectrolyte layers, for example alternating positively charged polyelectrolyte layers and negatively charged polyelectrolyte layers.

The multi-layer coating demonstrated higher stability to PCR denaturation temperatures.

The polyelectrolyte coating may be applied by simple dipping of the PCR substrate into a polyelectrolyte solution or it can be also applied by chemically reacting the polyelectrolyte with the PCR substrate to form covalent bonds that further strengthen the coating's stability to PCR conditions.

To this end, polyelectrolytes may be derivatized by introducing in the polymer chain terminations able to form covalent bonds with the surface making the film indefinitely stable during PCR.

The termination may be introduced during the copolymerization or alternatively, through direct reaction of the ionic groups of a preformed polyelectrolyte and the reactive unit of a bifunctional linker.

For example, polyacrylic acid polymers may be derivatized with the introduction of (trimethoxysilyl)propyl methacrylate monomers to form polymers having the following general formula I:

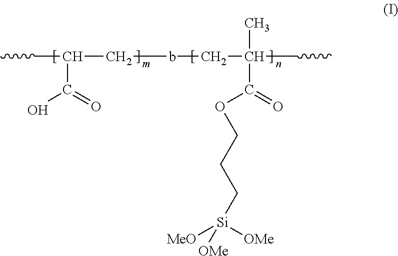

The polyelectrolyte of formula (I) may be used, for example, to coat a silicon dioxide passivated substrate, thus providing one or more additional passivation layers.

Another possible derivatization can be the addition of aminosilane groups to form polyelectrolytes containing monomers of formula II:

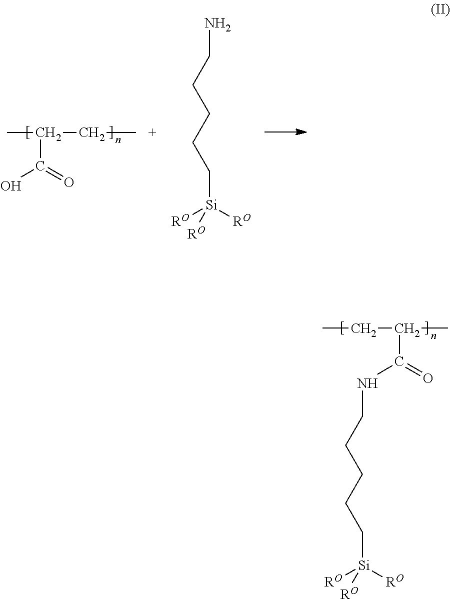

wherein R is an alkyl group. This polyacrylic acid derivative of formula (II) can be used for coating a silicon dioxide or glass substrate.

Further polyelectrolyte derivatives for silicon dioxide substrates can be obtained by reaction of the polyelectrolyte with a linker of formula III:

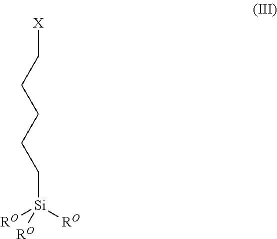

wherein R is an alkyl group and X is selected from Cl, Br, I, F or COCl.

Further polyelectrolyte derivatives for silicon substrates can be obtained by reaction of the polyelectrolyte with a linker of formula IV or V:

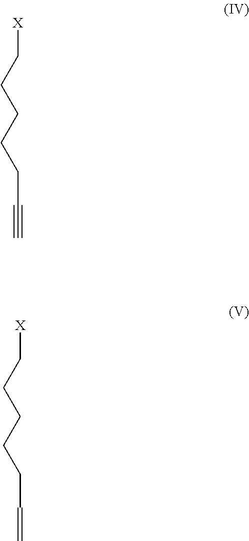

wherein X is selected from Cl, Br, I, F or COCl, ∥ is a double bond and [symbol] is a triple bond.

The polyelectrolyte coating may be applied to completely cover the PCR substrate or alternatively may be spotted onto controlled regions of the PCR substrate creating extremely hydrophilic, high liquid confining regions due to the high wettability of these compounds with respect to the PCR substrates. This allows a precise confining of the PCR mix onto predefined regions avoiding cross-contamination of samples on the same PCR substrate.

According to a further embodiment, a further increase of the difference in wettability between the polyelectrolyte coating and the PCR substrate may be obtained by covering with a hydrophobic polymer layer those regions of the PCR substrate not covered with the polyelectrolyte coating so as to delimit and confine the hydrophilic regions and creating hydrophobic regions. In particular, the hydrophobic polymer layer may be selected from the group consisting of polydimethylsiloxane (PDMS), polytetrafluoroethylene (PTFE), polyvynyldifluoride (PVDF), polycarbonate (PC), polyethylene (PE), polypropylene (PP) and polyimide.

In this case a very high contact angle difference can be obtained between the polyelectrolyte coating and its borders covered with the hydrophobic polymer allowing the maximum confining strength toward to PCR mix aqueous solutions. This has great importance when performing highly parallelized PCR tests due to the possible sample cross-contamination among close areas, for example, during reagents loading.

According to a second aspect of the present invention, it is further provided a method for passivating a PCR substrate comprising the step of applying a polyelectrolyte coating on a PCR substrate.

### BRIEF DESCRIPTION OF THE DRAWINGS

The invention will be now described with reference to the figures wherein:

FIG. 1 illustrates a first embodiment of the device according to the invention;

FIG. 2 illustrates a second embodiment of the device according to the invention;

FIG. 3 illustrates a third embodiment of the device according to the invention;

FIG. 4 illustrates a fourth embodiment of the device according to the invention;

FIG. 5 illustrates a method of preparation of the device according the present invention.

### DESCRIPTION OF EMBODIMENTS OF THE INVENTION

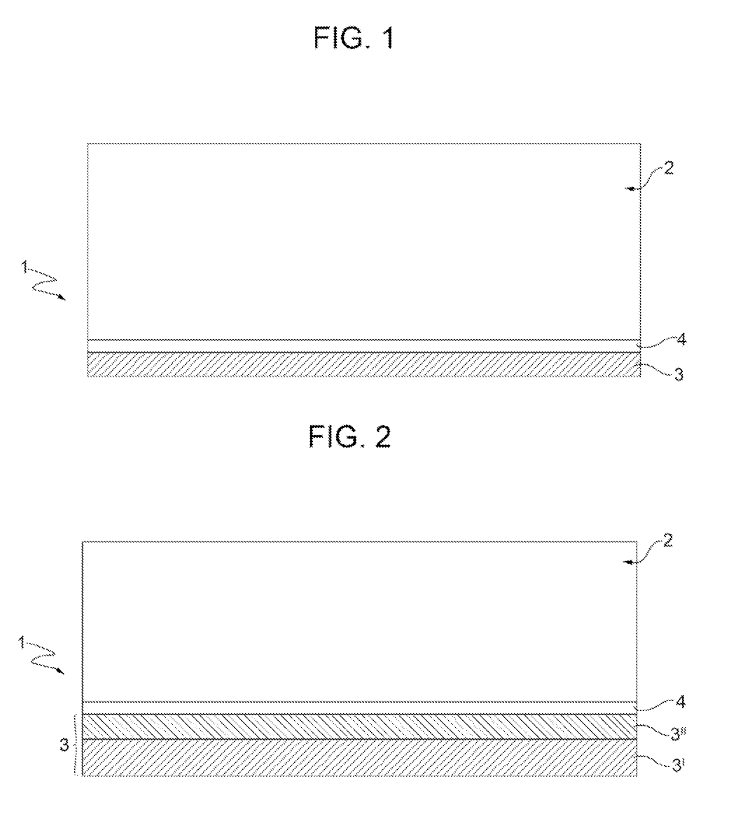

In FIG. 1, the PCR device 1 comprises a chamber 2 defining a reaction environment delimited at the bottom by a silicon substrate 3. The silicon substrate 3 is coated with a polyacrylic acid coating 4.

Alternately, the substrate may be a silicon-dioxide passivated substrate, as illustrated in FIG. 2, wherein the PCR substrate 3 comprises a silicon substrate 3′ passivated with a silicon dioxide layer 3″. The PCR substrate 3 is then covered with a polyelectrolyte coating 4.

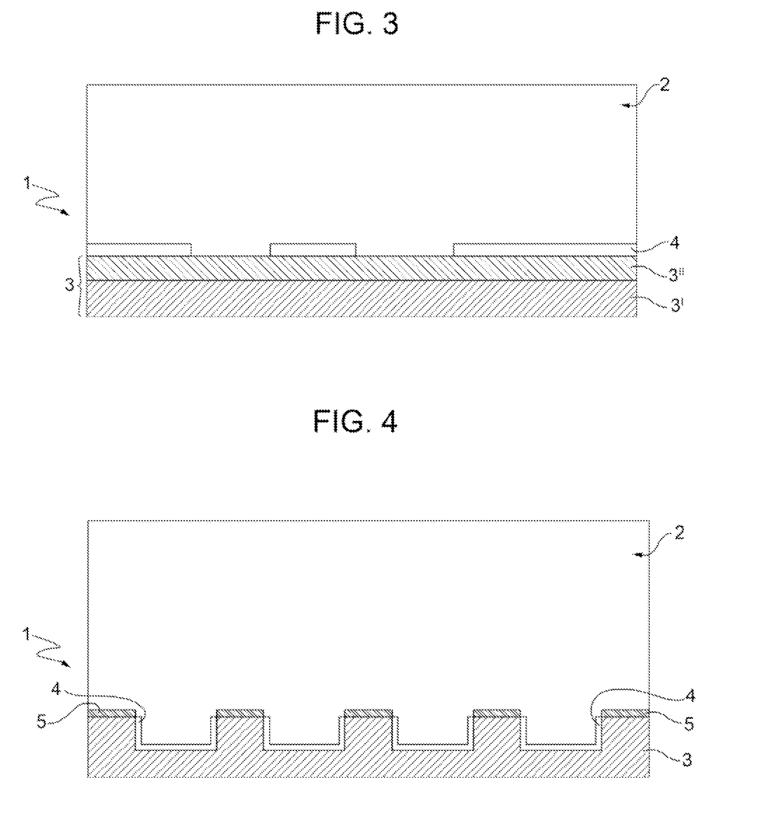

In a further embodiment, illustrated in FIG. 3, the PCR device 1 comprises a chamber 2 delimited by a PCR substrate 3 comprising a silicon substrate 3′ passivated with a silicon dioxide layer 3″. The PCR substrate 3 is spotted with a polyacrylic acid coating 4 in controlled regions.

FIG. 4 shows a fourth embodiment of the invention. A silicon substrate 3 is covered with a thin spin coated PDMS layer and subsequently etched by means of anisotropic plasma process to give an array of few micrometers to hundreds micrometers deep vessels 6. A polyacrylic acid coating 5 is applied into the vessels 6.

For a better understanding of the present invention a preferred embodiment thereof is now described, purely by way of a non-limiting example.

### Example 1

### Compatibility of Polyelectrolytes with PCR Mix Components

To evaluate the compatibility of the polyelectrolyte coating with PCR mix components, the inhibition of PCR was evaluated directly in a PCR mix. 0.5 μL of water solution of polyacrylic acid (PAA) at 8×10−3 M was dissolved into 50 μL of conventional PCR mix.

Different DNA dilutions were tested, from 300 pg to 3 fg of DNA. A 2% agarose gel was used to separate the amplified DNA fragments. Other tests with fast PCR-mix (20 μL) and RT-PCR were also carried out.

In every case (data not shown), no inhibition was observed, indicating that polyacrylate is completely compatible with the PCR reaction.

### Example 2

### Contact Angle Measurement on a Silanized PCR Substrate Spotted with Polyelectrolyte

A polyacrylic acid solution is spotted onto a silanized silicon substrate to obtain controlled regions with dimensions matching squared RT-PCR chambers of 4×4 mm. Contact angle measurements were obtained with 2 μL drops of water. The contact angle of the drop on the silanized surface was 56°, whereas the contact angle of the drop on polyacrylic acid region was only 5°, indicating that the polyacrylic acid coating greatly increased the wettability of the surface. Moreover, the difference in wettability caused the water to remain confined inside polyacrylic acid region, even after inclination (tilting) of the substrate.

### Example 3

### Contact Angle Measurement on a PDMS Coated PCR Substrate Spotted with Polyelectrolyte

After cleaning the silicon chip, a PDMS solution containing PDMS and a curing agent in a proportion 10:1 was deposited by a spin coating technique using different angular speeds with SCS P6700 (Specialty Coating Systems, Ind.). PDMS thickness measurements were performed by surface scratch and stylus profilometer technique.

The PDMS coated chip (about 20 micrometers) was then etched with a CF4/O2 (3:1) plasma, 270 W RF power, gas pressure 47 mTorr, etch rate 0.5 μm/min and subsequently a PAA coating was spotted on the etched surfaces.

Contact angles on the chip were then measured, as above. The results obtained were θ=10° for PAA regions and θ=120° for PDMS regions. Very high contact angle difference between the polyelectrolyte area and its borders allows the maximum confining strength toward water solutions. This is of great importance when performing high parallelized PCR tests, as the hydrophobic borders surrounding the hydrophilic reaction areas serves to contain the PCR solutions and prevent cross-contamination.

The inventors have exemplified the passivation coatings and methods with PCR reaction mixtures, but these coatings and methods can be applied to any chemical or biological analyses where the same reactivity issues are presented.

### Example 4

### Temperature Resistance

Three PCR devices having a multi-layer polyelectrolyte coating have been prepared.

Silicon chips were cleaned with a 1M NaOH solution for 5 minutes and then rinsed with deionized water. Immediately, an oxygen plasma treatment was carried out using Sentech 591 Reactive Ion Etching System (Sentech Instruments GmbH) under the following conditions: 60 sccm O2, 120 seconds, 16 Pa, 30 Watt RF.

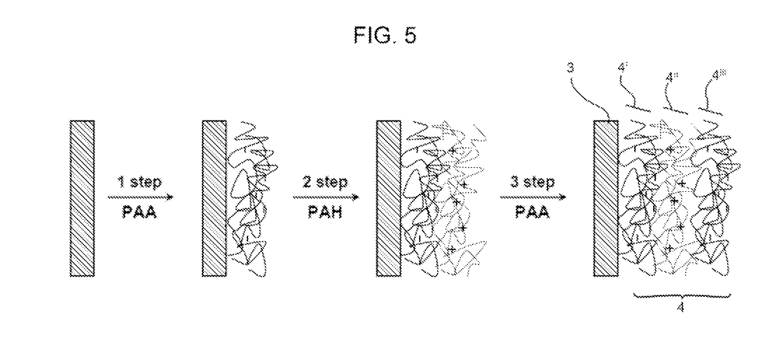

After cleaning the chips, a layer-by-layer deposition of polyacrylic acid (PAA) and polyallylamine (PAH) was carried out on two of them by dipping the chips alternately in baths of the two polyelectrolytes in a concentration 0.02M for 5 minutes as illustrated in FIG. 5. After each dipping, each chip was rinsed with water for 1 minute and dried in nitrogen flux.

The chips obtained were exposed to a temperature of 100° C. for 1 h to simulate the resistance of the chip to about 30 thermal cycles of amplification.

Table 1 shows contact angles of the chips measured after the thermal treatment compared with those obtained before the thermal treatment. Measurements of the static contact angle have been carried out with a CAM 200 (KSV Instruments Ltd) using 1 μl of Millipore water. The reported values are mean values.

TABLE 1

| | | Contact angles before and | |
|---|---|---|---|
| | | after thermal treatment | |
| | Process | θ₀ | θₜ |
| Chip 1 | cleaning | 25° | 35° |
| Chip 2 | Cleaning/functionalization | ~0° | ~0° |
| Chip 3 | Cleaning/functionalization | ~0° | ~0° |

As can be seen, the contact angle measured on the naked silicon chip after cleaning denotes a low wettability of the surface, whereas, after the functionalization with the polyelectrolyte coating, the contact angle of the PCR substrate cannot be measured to indicate that the substrate is completely wettable before the thermal treatment and maintains its properties also after heat treatment.

What is claimed is:

1. A PCR device comprising at least a chamber, said chamber being delimited on one side by a PCR substrate at least partially covered by a polyelectrolyte coating forming hydrophilic regions, said polyelectrolyte coating comprising at least one layer, said layer facing said chamber and being a negatively charged polyelectrolyte layer.

2. The PCR device according to claim 1, wherein said negatively charged polyelectrolyte layer is selected from the group consisting of polyacrylic acid, polymethacrylic acid, poly(glutamic acid), polystyrene sulfonate, derivatives, mixtures and copolymers thereof.

3. The PCR device according to claim 2, wherein said polyelectrolyte is polyacrylic acid.

4. The PCR device according to claim 1, wherein said polyelectrolyte coating comprises at least a further layer between said PCR substrate and said negatively charged layer, said further layer being a polyelectrolyte layer selected from the group consisting of polyacrylic acid, polyallylamine hydrochloride, polymethacrylic acid, polylysine, poly(glutamic acid), polystyrene sulfonate, polydiallyldimethyl-ammonium chloride, derivatives, mixtures and co-polymers thereof.

5. The PCR device according to claim 1, wherein said PCR substrate is selected from the group consisting of a semiconductor substrate, a metal substrate, a glass substrate and a polymeric substrate.

6. The PCR device according to claim 5, wherein said semiconductor substrate is selected from the group consisting of a silicon substrate, a silicon nitride passivated substrate, a silicon dioxide passivated substrate and a silanized substrate.

7. The PCR device according to claim 1, wherein said polyelectrolyte coating completely covers said PCR substrate.

8. The PCR device according to claim 1, wherein said PCR substrate is partially covered by a layer of hydrophobic polymer delimiting said hydrophilic regions and defining hydrophobic regions, said layer of hydrophobic polymer facing said chamber.

9. The PCR device according to claim 8, wherein said hydrophobic polymer is selected from the group consisting of polydimethylsiloxane (PDMS), polytetrafluoroethylene (PTFE), polyvynyldifluoride (PVDF), polycarbonate (PC), polyethylene (PE), polypropylene (PP) and polyimide.

10. A method for passivating a PCR substrate comprising the step of applying a polyelectrolyte coating on said PCR substrate.

11. A method for passivating a PCR substrate according to claim 10, wherein said polyelectrolyte coating is a polyacrylic acid layer.

12. A PCR device comprising a semiconductor substrate having at least one chamber,
said chamber partially or completely covered by at least one negatively charged polyelectrolyte coating forming at a plurality of hydrophilic regions, said hydrophilic regions being surrounded by a coating of a hydrophobic polymer to delimit said hydrophilic regions and define hydrophobic regions therebetween,
wherein said polyelectrolyte coating is selected from the group consisting of polyacrylic acid, polyallylamine hydrochloride, polymethacrylic acid, polylysine, poly(glutamic acid), polystyrene sulfonate, polydiallyldimethyl-ammonium chloride, derivatives, mixtures and copolymers thereof;
wherein said hydrophobic polymer is selected from the group consisting of polydimethylsiloxane, polytetrafluoroethylene, polyvynyldifluoride, polycarbonate, polyethylene, polypropylene, polyimide, derivatives, mixtures and copolymers thereof.

13. A method for passivating a PCR substrate comprising the steps of:
applying at least one negatively charged polyelectrolyte coating on said PCR substrate, wherein said polyelectrolyte coating is selected from the group consisting of polyacrylic acid, polyallylamine hydrochloride, polymethacrylic acid, polylysine, poly(glutamic acid), polystyrene sulfonate, polydiallyldimethyl-ammonium chloride, derivatives, mixtures and copolymers thereof, and
optionally surrounding areas of polyelectrolyte coating with a coating of said hydrophobic polymer selected from the group consisting of polydimethylsiloxane, polytetrafluoroethylene, polyvynyldifluoride, polycarbonate, polyethylene, polypropylene, polyimide, derivatives, mixtures and copolymers thereof.
